SEQUENCE LISTING (2 sequences)

SEQ ID NO: 1 - protein, 42 aa, synthetic construct [sequence omitted]

SEQ ID NO: 2 - protein, 40 aa, synthetic construct
DAEFRHDSGYEVHHQKLVFFAEDVGSNKGAIIGLMVGGVV

(19) United States
(12) Patent Application Publication
(10) Pub. No.: US 2024/0012002 A1
(43) Pub. Date: Jan. 11, 2024

(54) COUPLING LASER CAPTURE MICRODISSECTION WITH MICROFLUIDIC SAMPLE PREPARATION AND MASS SPECTROMETRY

(71) Applicants: Alexandra Ros, Phoenix, AZ (US); Jorvani Cruz Villarreal, Tempe, AZ (US); Ana Egatz-Gomez, Phoenix, AZ (US); Todd Sandrin, Phoenix, AZ (US); Paul Coleman, Fountain Hills, AZ (US)

(72) Inventors: Alexandra Ros, Phoenix, AZ (US); Jorvani Cruz Villarreal, Tempe, AZ (US); Ana Egatz-Gomez, Phoenix, AZ (US); Todd Sandrin, Phoenix, AZ (US); Paul Coleman, Fountain Hills, AZ (US)

(21) Appl. No.: 18/335,713

(22) Filed: Jun. 15, 2023

Related U.S. Application Data
(60) Provisional application No. 63/358,562, filed on Jul. 6, 2022.

(51) Int. Cl. G01N 33/68; G01N 1/28; G01N 33/53; B01L 3/00

(52) U.S. Cl. CPC G01N 33/6851; G01N 1/28; G01N 33/5304; B01L 3/502738; G01N 2001/284; B01L 2300/0816; B01L 2300/049; B01L 2400/0638; G01N 2333/46

(57) ABSTRACT

Described herein are systems and methods for a microfluidic immunoassay for in situ mass spectrometry analysis of intracellular protein biomarkers in tissue. In some embodiments, the tissue may comprise human brain tissue. In some embodiments, the protein biomarkers may comprise Aβ species comprising monomers and oligomers of Aβ₁₋₄₂, Aβ₁₋₄₀, Aβ₁₋₃₉, Aβ₂₋₄₃, or combinations thereof. In some embodiments, the systems and methods may comprise laser capture microdissection (LCM) and matrix-assisted laser desorption/ionization (MALDI) mass spectrometry.

### CROSS-REFERENCE TO RELATED APPLICATIONS

This application claims priority to U.S. Provisional Patent Application No. 63/358,562, filed on Jul. 6, 2022, which is incorporated by reference herein in its entirety.

### FEDERALLY SPONSORED RESEARCH

This invention was made with government support under R21 AG067488 awarded by the National Institutes of Health. The government has certain rights in the invention.

### REFERENCE TO SEQUENCE LISTING

This application was filed with a Sequence Listing XML in ST.26 XML format accordance with 37 C.F.R. § 1.831. The Sequence Listing XML file submitted in the USPTO Patent Center, “208192-9121-US02_sequence_listing_30-MAY-2023.xml,” was created on May 30, 2023, contains 2 sequences, has a file size of 2.93 Kbytes, and is incorporated by reference in its entirety into the specification.

### TECHNICAL FIELD

Described herein are systems and methods for a microfluidic immunoassay for in situ mass spectrometry analysis of intracellular protein biomarkers in tissue. In some embodiments, the tissue may comprise human brain tissue. In some embodiments, the protein biomarkers may comprise Aβ species comprising monomers and oligomers of Aβ₁₋₄₂, Aβ₁₋₄₀, Aβ₁₋₃₉, Aβ₂₋₄₃, or combinations thereof. In some embodiments, the systems and methods may comprise laser capture microdissection (LCM) and matrix-assisted laser desorption/ionization (MALDI) mass spectrometry.

### BACKGROUND

Alzheimer's Disease (AD) is a neurodegenerative disorder characterized by the aggregation of amyloid-β peptide (Aβ) and tau protein. Protein aggregation starts decades before most individuals present any dementia symptoms. Although the pathophysiology of AD is still not well understood, a well-supported hypothesis suggests that soluble Aβ oligomers (aggregate intermediates along the formation of plaques) are the main neurotoxic species involved in the pathological cascade of the disease, however, their role is yet to be fully established. An improved understanding of the Aβ species and their abundance in neurons will contribute to elucidating the role of Aβ in AD, developing therapeutic and diagnostic tools, and advancing the cure of this devastating disease.

Aβ peptides are the proteolytic products of the amyloid protein precursor (APP), cleaved into fragments with amino acid lengths ranging from 37 to 43 residues. It has been proposed that AD is caused by an imbalance between Aβ production and clearance. Aβ aggregates in the extracellular matrix into plaques. However, the soluble Aβ species that can localize intracellularly are among the likely culprits for the cognitive impairment and neurotoxicity in AD pathogenesis. A wealth of data is available about Aβ through in vitro studies and animal models; however, given the complexity of the human nervous system, assessment of oligomeric Aβ in the human brain can provide information more relevant to human AD. Clinically annotated human brain samples from tissue banks represent a golden opportunity to perform such studies.

Immunohistochemistry and immunofluorescence methods can effectively localize proteins in tissue, but they are semi-quantitative techniques and depend on the availability of tags for target molecules. Mass spectrometry (MS) approaches allow for quantitative unbiased studies and biomolecule identification. Brain homogenates, widely used in combination with immunoprecipitation (IP) and MS analysis, such as liquid chromatography (LC) MS/MS, electrospray ionization (ESI) MS, or matrix-assisted laser desorption ionization (MALDI) MS, do not provide information with spatial resolution. Fortunately, MS can be performed with microscopic amounts of tissue. MALDI MS Imaging (MALDI MSI) is a widely used technique for tissue protein profiling, which provides label-free detection and mapping of multiple analytes. Limitations in MALDI MSI may arise for low abundant analytes, which can be masked by other highly abundant species. In addition, MALDI MSI often requires confirmation by other techniques such as LC-MS.

Laser capture microdissection (LCM) allows for selectively excising single cells from specific tissue regions of interest. This method has been widely used in single-cell genomics. Yet, proteomics can provide information on phenotype, post-translational modifications, protein concentration, and protein-protein interactions. While there are technologies to address the genome and transcriptome of single cells, there is still a lack of technology for quantitative analysis of the intracellular protein content of specific subpopulations in tissues. The proteomic analysis of small cell populations must avoid sample-processing dilution and loss of precious minute analyte amounts. However, by assessing the entire proteome, low-abundance proteins, as is the case of intracellular Aβ oligomers, can be masked by high-abundance ones. A combination of nanoliter droplet arrays with MALDI-MS was used for the analysis of proteins secreted by encapsulated cells. This approach, however, is inadequate for the assessment of the intracellular content of individual tissue cells.

What is needed are novel systems and methods for a microfluidic immunoassay for in situ mass spectrometry analysis of intracellular Aβ species in tissue, such as human brain tissue.

### SUMMARY

One embodiment described herein is a method for analyzing tissue for the presence of Aβ-M and Aβ-O species, the method comprising: providing a sample of tissue comprising cells; microdissecting the cells and transferring the cells to an upper chamber of a manifold comprising a plurality of layered wells each comprising an upper chamber and a lower chamber, each chamber comprising independent fluidic connections and an adjustable valve separating the upper chambers and lower chambers; assembling the manifold on an indium-titanium oxide coated glass slide; introducing one or more anti-Aβ antibodies into the lower chamber of the layered well containing the cells in the upper chamber, incubating for a period of time, and washing the layered well; opening the adjustable valve separating the upper chamber and lower chamber to permit the cells in the upper chamber to contact the one or more anti-Aβ antibodies in the lower chamber, incubating for a period of time, and washing the layered well to remove non-captured material; introducing a matrix solution and allowing crystallization; and removing the manifold and analyzing a co-crystallized sample using mass spectrometry to identify the presence of the Aβ-M and Aβ-O species. In one aspect, the tissue is human brain tissue comprising human brain cells. In another aspect, the manifold is comprised of a polymeric material comprising poly(dimethylsiloxane) (PDMS), polycarbonate (PC), poly-methyl-meta-acrylate (PMMA), cyclic olefin copolymer (COC), polyimide, or combinations thereof. In another aspect, the manifold is comprised of PDMS. In another aspect, the one or more anti-Aβ antibodies comprises an Aβ specific antibody, an amyloid oligomer-specific antibody, or a combination thereof. In another aspect, the one or more anti-Aβ antibodies comprises an immunoglobulin G (IgG) 6E10 antibody. In another aspect, the matrix solution comprises α-cyano-4-hydroxycinnamic acid or sinapinic acid in acetonitrile and trifluoroacetic acid. In another aspect, the mass spectrometry comprises matrix-assisted laser desorption/ionization (MALDI) mass spectrometry. In another aspect, each layered well comprises a well area size ranging from about 50 μm×about 50 μm to about 500 μm×about 500 μm. In another aspect, each layered well comprises a well area size of about 500 μm×about 500 μm. In another aspect, microdissecting the cells comprises laser capture microdissection (LCM). In another aspect, each layered well comprises from about 1 to about 100 individual cells. In another aspect, each layered well comprises from about 1 to about 20 individual cells. In another aspect, the Aβ-M species comprise monomers of Aβ₁₋₄₂, Aβ₁₋₄₀, Aβ₁₋₃₉, Aβ₂₋₄₃, or combinations thereof. In another aspect, the Aβ-O species comprise oligomers of Aβ₁₋₄₂, Aβ₁₋₄₀, Aβ₁₋₃₉, Aβ₂₋₄₃, or combinations thereof. In another aspect, the oligomers of Aβ₁₋₄₂, Aβ₁₋₄₀, Aβ₁₋₃₉, Aβ₂₋₄₃, or combinations thereof comprise dimers, trimers, tetramers, pentamers, hexamers, heptamers, octamers, nonamers, decamers, 11-mers, 12-mers, 13-mers, 14-mers, 15-mers, 16-mers, 17-mers, 18-mers, 19-mers, 20-mers, or combinations thereof. In another aspect, the method further comprises a bovine serum albumin (BSA) blocking step in the layered well prior to opening the adjustable valve. In another aspect, the method has a limit of detection for the Aβ-M and Aβ-O species of about 1.60×10⁸ to about 2.90×10¹¹ Aβ molecules per layered well.

Another embodiment described herein is a system for analyzing tissue for the presence of Aβ-M and Aβ-O species, the system comprising: an apparatus for microdissection of cells from a sample of tissue; a manifold comprising a plurality of layered wells each comprising an upper chamber and a lower chamber, each chamber comprising independent fluidic connections and an adjustable valve separating the upper chambers and lower chambers, wherein the manifold is assembled on an indium-titanium oxide coated glass slide; one or more anti-Aβ antibodies positioned within the lower chamber of the layered well; a matrix solution; and a mass spectrometer. In one aspect, the sample of tissue is a sample of human brain tissue comprising human brain cells. In another aspect, the apparatus for microdissection comprises a laser capture microdissection (LCM) apparatus. In another aspect, the manifold is comprised of a polymeric material comprising poly(dimethylsiloxane) (PDMS). In another aspect, the one or more anti-Aβ antibodies comprises an Aβ-specific antibody, an amyloid oligomer-specific antibody, or a combination thereof. In another aspect, the one or more anti-Aβ antibodies comprises an immunoglobulin G (IgG) 6E10 antibody. In another aspect, the matrix solution comprises a cyano-4-hydroxycinnamic acid or sinapinic acid in acetonitrile and trifluoroacetic acid. In another aspect, the mass spectrometer comprises a mass spectrometer configured for matrix-assisted laser desorption/ionization (MALDI) mass spectrometry.

Another embodiment described herein is a method for analyzing tissue for the presence of one or more protein biomarkers, the method comprising: providing a sample of tissue comprising cells; microdissecting the cells and transferring the cells to an upper chamber of a manifold comprising a plurality of layered wells each comprising an upper chamber and a lower chamber, each chamber comprising independent fluidic connections and an adjustable valve separating the upper chambers and lower chambers; assembling the manifold on an indium-titanium oxide coated glass slide; introducing one or more antibodies into the lower chamber of the layered well containing the cells in the upper chamber, incubating for a period of time, and washing the layered well; opening the adjustable valve separating the upper chamber and lower chamber to permit the cells in the upper chamber to contact the one or more antibodies in the lower chamber, incubating for a period of time, and washing the layered well to remove non-captured material; introducing a matrix solution and allowing crystallization; and removing the manifold and analyzing a co-crystallized sample using mass spectrometry to identify the presence of the one or more protein biomarkers.

### DESCRIPTION OF THE DRAWINGS

FIG. 1A-D show MIMAS schematics. FIG. 1A shows a microfluidic device top view. The fluidic layer is filled in solid gray. The control layer is marked with dotted lines. Inset, top: cross-sections of a normally closed valve. Inset, bottom: a valve opened by vacuum action, upon which two adjacent wells become connected. FIG. 1B shows a cross-section of a collection-layer funnel-shaped well, aligned over an upward-facing MIMAS device well. FIG. 10 shows a cross-section of a MIMAS device assembled on an ITO-coated glass slide. FIG. 1D shows analyte-matrix co-crystals over an ITO-coated glass slide after MIMAS manifold removal, being ionized during MALD I-MS.

FIG. 2A-D show the Collection Layer Fabrication. FIG. 2A shows a collection layer schematic. (1) the NOA-81 mold is placed and secured over a glass slide. (2) PDMS is poured over the mold and cured. (3) The PDMS layer is removed from the mold. (4) The layer is placed over the MIMAS device, aligning the bottom squares with the wells on the MIMAS top (fluidic) layer. FIG. 2B shows a computer-aided design (CAD) of the collection layer mold using Fusion360. FIG. 2C shows from left to right: microscopy images of the IP-S mold, the NOA-81 mold, and the PDMS collection layer. A positive funnel is first 3D printed, then the negative is formed with this first mold in NOA-81, and finally a positive funnel is formed in PDMS from the NOA-81 mold. Scale bar: 500 μm. FIG. 2D shows a CAD image of the collection layer aligned over the MIMAS fluidic layer wells.

FIG. 3A-B show collection of microdissected brain cells. Dissection schematics and representative microscopy images of collected cells into a 500 μm×500 μm MIMAS well (FIG. 3A-1, indicated by dashed lines), some cells are found outside the well. FIG. 3A-2 shows a 2 mm-diameter milli-well, and FIG. 3A-3 shows a 1 mm-diameter collection layer funnel on a MIMAS well. FIG. 3B shows cell capture efficiency for FIG. 3A-1, FIG. 3A-1, and FIG. 3A-3; the error bars represent the standard deviation.

FIG. 4A-F show Aβ monomer immunocapture in milli-wells. FIG. 4A shows schematics of conditions using Aβ₁₋₄₀ or Aβ₁₋₄₂ with IgG 6E10 and a non-binding IgG. FIG. 4B shows Aβ42 experiment performed with a non-binding IgG where no Aβ peak was observed. FIG. 4C shows Aβ₁₋₄₀ control experiment without antibody immobilized showing no Aβ peak. FIG. 4D shows Aβ₁₋₄₀ immunocaptured by IgG 6E10 showing [Aβ+H]⁺ at m/z 4,330 and [Aβ+2H]²⁺ at m/z 2,165. FIG. 4E shows Aβ₁₋₄₂ control experiment without antibody immobilized showing no Aβ peak. FIG. 4F shows Aβ₁₋₄₂ immunocaptured by IgG 6E10 showing [Aβ+H]⁺ at m/z 4,514 and [Aβ+2H]²⁺ at m/z 2,257. Monomeric Aβ concentration=1 μM.

FIG. 5A-B show Aβ oligomer immunocapture using milli-wells. FIG. 5A shows Aβ₁₋₄₀-O immunocaptured with IgG 6E10: Aβ₁₋₄₀ monomers, dimers, tetramers, and pentamers are observed. Peaks with m/z ˜4720 and m/z ˜8570 are labeled as U1 and U2, respectively. FIG. 5B shows Aβ₁₋₄₂-O immunocaptured by IgG 6E10: Aβ₁₋₄₂ monomers, dimers, and tetramers. Peaks with m/z ˜4720 and m/z ˜8570 are labeled as U3 and U4, respectively. The thick white arrows indicate peaks related to the blocking step. Aβ concentration (monomeric species)=1 μM. Concentration of oligomer species is unknown.

FIG. 6A-B show representative MS spectra of Aβ₁₋₄₀ and Aβ₁₋₄₂ monomers at low concentrations. FIG. 6A shows a spectrum of a solution of 50 nM Aβ₁₋₄₀ monomers with a S/N of 8. FIG. 6B shows a spectrum of a solution of 100 nM Aβ₁₋₄₂ monomers with a S/N of 12.

FIG. 7A-B show Aβ-M IgG 6E10 immunocapture using the MIMAS platform. FIG. 7A shows Aβ₁₋₄₀-M immunocapture exhibits [Aβ+H]⁺ at m/z 4,330 and [Aβ+2H]²⁺ at m/z 2,165. FIG. 7B shows Aβ₁₋₄₂-M immunocapture exhibits [Aβ+H]⁺ at m/z 4,514 and [Aβ+2H]²⁺ at m/z 2,257. The arrows indicate the m/z ˜3,880 peak associated with the BSA blocking step. The Aβ species concentration was 200 nM in each case.

FIG. 8 shows immunoassay performed on MIMAS wells using Aβ₁₋₄₂ at various concentrations. The bars represent the standard deviation (n=15). The line represents the linear fit with R²=0.84.

FIG. 9A-B show standard curves of Aβ₁₋₄₀ and Aβ₁₋₄₂ using milli-wells. Average S/N of MALDI-MS analysis of Aβ₁₋₄₀ (FIG. 9A) and Aβ₁₋₄₂ (FIG. 9B) at various concentrations. The bars represent the standard deviation (n=15). The dashed line represents the linear fit with R²=0.98 for FIG. 9A and R²=0.96 for FIG. 9B.

FIG. 10A-B show standard curves of Aβ₁₋₄₀ and Aβ₁₋₄₂ using the MIMAS platform. Average S/N of MALDI-MS analysis of Aβ₁₋₄₀ (FIG. 10A) and Aβ₁₋₄₂ (FIG. 10B) at various concentrations. The bars represent the standard deviation (n=15). Dashed lines represent the linear fitting with 0.98 for FIG. 10A and R²=0.98 for FIG. 10B.

FIG. 11A-B show Aβ oligomer IgG 6E10 immunocapture in the MIMAS platform. FIG. 11A shows Aβ₁₋₄₀-O immunocaptured monomers, dimers, and tetramers. Peaks with m/z ˜4720 and m/z ˜8570 are labeled U1 and U2. FIG. 11B shows Aβ₁₋₄₂-O immunocaptured monomers, dimers, and tetramers. Peaks with m/z ˜4720 and m/z ˜8570 are labeled U3 and U4. The thick white arrows indicate peaks related to the blocking step. Aβ concentration (prior oligomerization)=1 μM.

FIG. 12A-B show MIMAS platform control experiments without immobilized antibody using without Aβ₁₋₄₀-M (FIG. 12A) and without Aβ₁₋₄₂-M (FIG. 12B) showing a lack of peaks related to the corresponding Aβ species. The thick white arrows indicate the m/z are where [Aβ+H]⁺ is expected for each case: Aβ₁₋₄₀ at m/z 4,330 and Aβ₁₋₄₂ at m/z 4,514.

FIG. 13A-C show immunoassay performed in milli-wells using brain cells. FIG. 13A shows representative MS spectrum resulting from the immunoassay in milli-wells using 100 brain slice cells. Sections marked as A and B are shown zoomed-in in FIG. 13B and FIG. 13C, respectively. FIG. 13B shows a zoomed-in spectrum of section A showing the peaks with m/z 4,270, m/z 4,374, and m/z 4,490. FIG. 13C shows zoomed-in of section B showing peaks with m/z 8570, m/z 8760, and m/z 8970.

FIG. 14 shows resulting spectrum of control Aβ oligomer experiments without IgG 6E10 at higher mass range. The observed peaks are associated to the blocking step performed with 1% BSA.

FIG. 15 shows schematics of the LCM-MIMAS workflow: (1) brain cell microdissection and loading into the microfluidic wells with the help of the collection layer, (2) removal of collection layer, (3) assembly of the MIMAS manifold with the collected cells in the wells of fluidic line I onto an ITO glass slide, (4) immobilization of antibodies and blocking step in fluidic line II, (5) mixing wells I and II contents and incubation for immunocapture, (6) wash to remove non-captured material, (7) matrix solution loading and mixing with the sample, (8) removal of the MIMAS elastomeric manifold to expose the sample-matrix crystals for MS analysis.

FIG. 16 shows analysis of Aβ in cells from archived brain tissue with the LCM-MIMAS approach. Representative MS spectrum from the MIMAS immunoassay, using a collection layer to load 20 brain tissue cells directly into MIMAS wells. Peaks m/z 4270.2 and m/z 8557.9 match with peaks observed in the milli-wells assay.

FIG. 17 shows resulting spectrum of control immunoassay using microdissected brain cells in milli-wells. Black arrows indicate the areas where peaks are observed when IgG 6E10 is immobilized on milli-wells and brain cells were loaded and treated as in the actual assay. There was no binding of Aβ species to the IgG.

### DETAILED DESCRIPTION

Unless otherwise defined, all technical and scientific terms used herein have the same meaning as commonly understood by one of ordinary skill in the art. For example, any nomenclatures used in connection with, and techniques of biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “subject” refers to an animal. Typically, the subject is a mammal. A subject also refers to primates (e.g., humans, male or female; infant, adolescent, or adult), non human primates, rats, mice, rabbits, pigs, cows, sheep, goats, horses, dogs, cats, fish, birds, and the like. In one embodiment, the subject is a primate. In one embodiment, the subject is a human. In one embodiment of the present invention, a tissue sample from a human subject is provided.

As used herein, the term “tissue” refers to any commonly known tissue of a mammal, such as a human. Tissue may include, but is not limited to, nervous tissue (e.g., brain tissue, spinal cord tissue, nerves, neuronal tissue), connective tissue (e.g., bone tissue, ligament tissue, tendon tissue, blood tissue, lymph tissue), epithelial tissue (e.g., skin surface tissue (epidermis), tissue lining of GI tract organs and other hollow organs), and muscle tissue (e.g., cardiac muscle tissue, smooth muscle tissue, skeletal muscle tissue). In some embodiments of the disclosed systems and methods, tissue may comprise human brain tissue.

As used herein, the terms “inhibit,” “inhibition,” or “inhibiting” refer to the reduction or suppression of a given biological process, condition, symptom, disorder, or disease, or a significant decrease in the baseline activity of a biological activity or process.

Described herein is a microfluidic assay for in situ mass spectrometry of immunocaptured Aβ species from archived human tissues. In this work, the previously reported microfluidic platform in tandem with MALDI mass spectrometry (MIMAS) was coupled with LCM to assess the Aβ protein content from tissue cells. See e.g., Cruz Villarreal et al., “MIMAS: microfluidic platform in tandem with MALDI mass spectrometry for protein quantification from small cell ensembles,” Anal. Bioanal. Chem. 414: 3945-3958 (2002); Yang et al., Quantitative Approach for Protein Analysis in Small Cell Ensembles by an Integrated Microfluidic Chip with MALDI Mass Spectrometry,” Anal. Chem. 93, 6053-6061 (2021), both of which are incorporated by reference herein in their entirety for their teachings. The LCM-MIMAS approach allows for selective dissection of individual cells from tissues, their transfer to the microfluidic platform for sample processing on-chip, and subsequent mass spectrometry identification and quantification. This work targets Aβ monomeric and oligomeric species from brain cells due to their relevance in the current amyloid hypothesis that soluble Aβ oligomers are responsible for neuronal dysfunction.

Also described herein is an approach that allows for the selective dissection of individual cells from tissues, their transfer to the microfluidic platform for sample processing on-chip, and mass spectrometric characterization. In one aspect, cells may be collected directly onto an integrated microfluidic assay in tandem with the MIMAS device. In another aspect, cells may be translocated to a device having a well area of about 500 μm×500 μm.

Three methods of cell collection were used in the described exemplary assays. In one aspect, cells were directly collected into milli-wells with 2 mm diameter wells. In another aspect, cells were directly collected into a MIMAS device. In another aspect, cells were collected into a collection layer fabricated in polydimethylsiloxane (PDMS) elastomer on top of the MIMAS device. Any cells or debris not translocated to the collection area were removed with the PDMS layer prior to further assembly steps. The misplaced dissected cells on device surfaces other than the MIMAS-wells could interfere with further assembly steps.

All experimental examples described herein with the MIMAS devices were performed using a removable PDMS collection layer in the cell collection step. In one non-limiting example, the MIMAS platform device assembly comprised an indium tin oxide (ITO) coated glass slide with a PDMS manifold. In this particular example, the PDMS manifold forming the MIMAS platform had two wells, one well with the protein of interest and one well with immobilized antibodies for capture. Each “fluidic line” contained five wells (i.e., 10 wells total), and pairs were formed from one well in each layer separated by a valve. Capture antibodies were then used for immunocapture of Aβ species and oligomers (e.g., IgG 6E10). The immunocapture steps involved adding the antibody, incubation, and washing steps. The captured Aβ species were removed from the chip and sent for MS analysis.

It should be understood that the described MIMAS platform is not to be restricted or limited to any particular number of wells. The example configurations described herein are only exemplary embodiments and are not meant to be limiting in any way. For example, the systems and methods described herein may comprise a manifold comprising a plurality of layered wells, wherein the plurality of layered wells may comprise from 2 wells to 1,000 wells or greater. In one embodiment, the manifold may comprise from 2 wells to 10 wells. In another embodiment, the manifold may comprise from 10 wells to 100 wells. In another embodiment, the manifold may comprise from 100 wells to 500 wells. In another embodiment, the manifold may comprise from 500 wells to 1,000 wells. In another embodiment, the manifold may comprise greater than 1,000 wells.

In some non-limiting aspects, the chip construction may comprise a fluidic layer with a thickness of about 25 μm, a PDMS mixture of about 15:1 w/w base to curing agent spin coated over a master wafer for the fluidic layer, creating a layer of about 63 μm thickness, double-layer PDMS slabs peeled-off, reservoirs punched using 2 mm biopsy punchers, and a removable collection layer added to facilitate the collection of cells.

One embodiment described herein is a method for analyzing tissue for the presence of Aβ-M and Aβ-O species, the method may comprise: providing a sample of tissue comprising cells; microdissecting the cells and transferring the cells to an upper chamber of a manifold comprising a plurality of layered wells each comprising an upper chamber and a lower chamber, each chamber comprising independent fluidic connections and an adjustable valve separating the upper chambers and lower chambers; assembling the manifold on an indium-titanium oxide coated glass slide; introducing one or more anti-Aβ antibodies into the lower chamber of the layered well containing the cells in the upper chamber, incubating for a period of time, and washing the layered well; opening the adjustable valve separating the upper chamber and lower chamber to permit the cells in the upper chamber to contact the one or more anti-Aβ antibodies in the lower chamber, incubating for a period of time, and washing the layered well to remove non-captured material; introducing a matrix solution and allowing crystallization; and removing the manifold and analyzing a co-crystallized sample using mass spectrometry to identify the presence of the Aβ-M and Aβ-O species.

In one aspect, the tissue may be human brain tissue comprising human brain cells.

In another aspect, the manifold may be comprised of a polymeric material comprising poly(dimethylsiloxane) (PDMS), polycarbonate (PC), poly-methyl-meta-acrylate (PMMA), cyclic olefin copolymer (COC), polyimide, or combinations thereof. In another aspect, the manifold may be comprised of PDMS.

In another aspect, the one or more anti-Aβ antibodies may comprise an Aβ-specific antibody, an amyloid oligomer-specific antibody, or a combination thereof. In another aspect, the one or more anti-Aβ antibodies may comprise an immunoglobulin G (IgG) 6E10 antibody. IgG 6E10 recognizes all species of Aβ without regard to conformation. Amyloid oligomer-specific antibodies may recognize all types of amyloid oligomers, but not monomers or fibrils. Any suitable Aβ-specific antibodies and/or amyloid oligomer-specific antibodies known in the art may be used in the systems and methods disclosed herein for analyzing tissue for the presence of Aβ-M and Aβ-O species.

In another aspect, the matrix solution may comprise α-cyano-4-hydroxycinnamic acid or sinapinic acid in acetonitrile and trifluoroacetic acid.

In another aspect, the mass spectrometry may comprise matrix-assisted laser desorption/ionization (MALDI) mass spectrometry.

In another aspect, each layered well may comprise a well area size ranging from about 50 μm×about 50 μm to about 500 μm×about 500 μm. In another aspect, each layered well may comprise a well area size of about 500 μm×about 500 μm. In some aspects, the sensitivity of the disclosed systems and methods may increase when smaller well area sizes are used. For example, systems and methods comprising well area sizes less than about 500 μm×about 500 μm may have a greater sensitivity as compared to systems and methods comprising well area sizes of about 500 μm×about 500 μm.

In another aspect, microdissecting the cells may comprise laser capture microdissection (LCM).

In another aspect, each layered well may comprise from about 1 to about 100 individual cells. In another aspect, each layered well may comprise from about 1 to about 20 individual cells. In some aspects, the number of cells in each layered well may depend on the specific tissue type analyzed.

In another aspect, the Aβ-M species comprise monomers of Aβ₁₋₄₂, Aβ₁₋₄₀, Aβ₁₋₃₉, Aβ₂₋₄₃, or combinations thereof. In another aspect, the Aβ-O species may comprise oligomers of Aβ₁₋₄₂, Aβ₁₋₄₀, Aβ₁₋₃₉, Aβ₂₋₄₃, or combinations thereof. In another aspect, the oligomers of Aβ₁₋₄₂, Aβ₁₋₄₀, Aβ₁₋₃₉, Aβ₂₋₄₃, or combinations thereof may comprise dimers, trimers, tetramers, pentamers, hexamers, heptamers, octamers, nonamers, decamers, 11-mers, 12-mers, 13-mers, 14-mers, 15-mers, 16-mers, 17-mers, 18-mers, 19-mers, 20-mers, larger oligomeric species, or combinations thereof. For example, in some aspects, the oligomers of Aβ₁₋₄₂, Aβ₁₋₄₀, Aβ₁₋₃₉, Aβ₂₋₄₃, or combinations thereof may comprise oligomeric species up to 30-mers, 40-mers, 50-mers, or larger.

In another aspect, the method further may comprise a bovine serum albumin (BSA) blocking step in the layered well prior to opening the adjustable valve.

In another aspect, the method may have a limit of detection for the Aβ-M and Aβ-O species of about 1.60×10⁸ to about 2.90×10¹¹ Aβ molecules per layered well.

Another embodiment described herein is a system for analyzing tissue for the presence of Aβ-M and Aβ-O species, the system may comprise: an apparatus for microdissection of cells from a sample of tissue; a manifold comprising a plurality of layered wells each comprising an upper chamber and a lower chamber, each chamber comprising independent fluidic connections and an adjustable valve separating the upper chambers and lower chambers, wherein the manifold is assembled on an indium-titanium oxide coated glass slide; one or more anti-Aβ antibodies positioned within the lower chamber of the layered well; a matrix solution; and a mass spectrometer.

In one aspect, the sample of tissue may be a sample of human brain tissue comprising human brain cells.

In another aspect, the apparatus for microdissection may comprise a laser capture microdissection (LCM) apparatus.

In another aspect, the manifold may be comprised of a polymeric material comprising poly(dimethylsiloxane) (PDMS).

In another aspect, the one or more anti-Aβ antibodies may comprise an Aβ-specific antibody, an amyloid oligomer-specific antibody, or a combination thereof. In another aspect, the one or more anti-Aβ antibodies may comprise an immunoglobulin G (IgG) 6E10 antibody. IgG 6E10 recognizes all species of Aβ without regard to conformation. Amyloid oligomer-specific antibodies may recognize all types of amyloid oligomers, but not monomers or fibrils. Any suitable Aβ-specific antibodies and/or amyloid oligomer-specific antibodies known in the art may be used in the systems and methods disclosed herein for analyzing tissue for the presence of Aβ-M and Aβ-O species.

In another aspect, the matrix solution may comprise α-cyano-4-hydroxycinnamic acid or sinapinic acid in acetonitrile and trifluoroacetic acid.

In another aspect, the mass spectrometry may comprise matrix-assisted laser desorption/ionization (MALDI) mass spectrometry.

Another embodiment described herein is a method for analyzing tissue for the presence of one or more protein biomarkers, the method comprising: providing a sample of tissue comprising cells; microdissecting the cells and transferring the cells to an upper chamber of a manifold comprising a plurality of layered wells each comprising an upper chamber and a lower chamber, each chamber comprising independent fluidic connections and an adjustable valve separating the upper chambers and lower chambers; assembling the manifold on an indium-titanium oxide coated glass slide; introducing one or more antibodies into the lower chamber of the layered well containing the cells in the upper chamber, incubating for a period of time, and washing the layered well; opening the adjustable valve separating the upper chamber and lower chamber to permit the cells in the upper chamber to contact the one or more antibodies in the lower chamber, incubating for a period of time, and washing the layered well to remove non-captured material; introducing a matrix solution and allowing crystallization; and removing the manifold and analyzing a co-crystallized sample using mass spectrometry to identify the presence of the one or more protein biomarkers.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

### EXAMPLES

### Example 1

Materials and Methods Aβ peptides, Aβ₁₋₄₂ (DAEFRHDSGYEVHHQKLVFFAEDVGSNKGAIIGLMVGGVVIA; SEQ ID NO: 1) and Aβ₁₋₄₀ (DAEFRHDSGYEVHHQKLVFFAEDVGSNKGAIIGLMVGGVV; SEQ ID NO: 2) were purchased from AnaSpec (USA), Ham's F-12 medium, from ThermoFisher (USA); isopropanol, ethanol, β-mercaptoethanol, and acetone, from VWR; SU-8 2075 photoresist and SU-8 developer from MicroChem (USA); acetonitrile (ACN), dimethyl sulfoxide (DMSO), hexafluoroisopropanol (HFIP), α-cyano-4-hydroxycinnamic acid (α-CHCA), sinapinic acid (SA), trifluoroacetic acid (TFA), and bovine serum albumin (BSA), from Sigma-Aldrich (USA); poly(dimethylsiloxane) (PDMS), from Dow Corning Corporation (USA); indium tin oxide (ITO)-coated glass slides (100 Ω/sq.), from Laser BioLabs (France); and NOA-81 UV curable epoxy, from Thorlabs, Inc. (USA).

### Microfluidic Platform Fabrication and Assembly

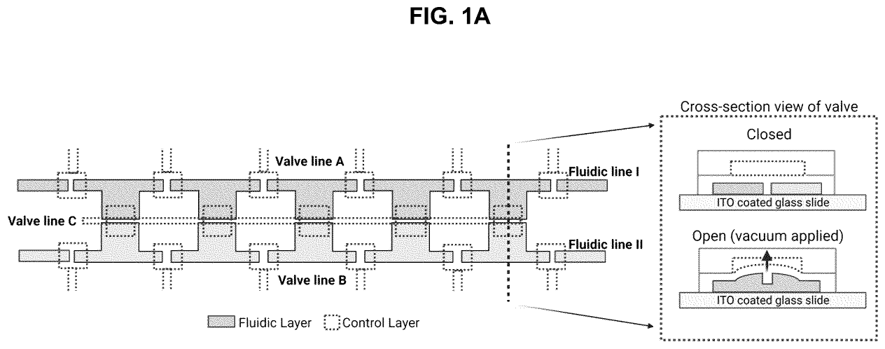

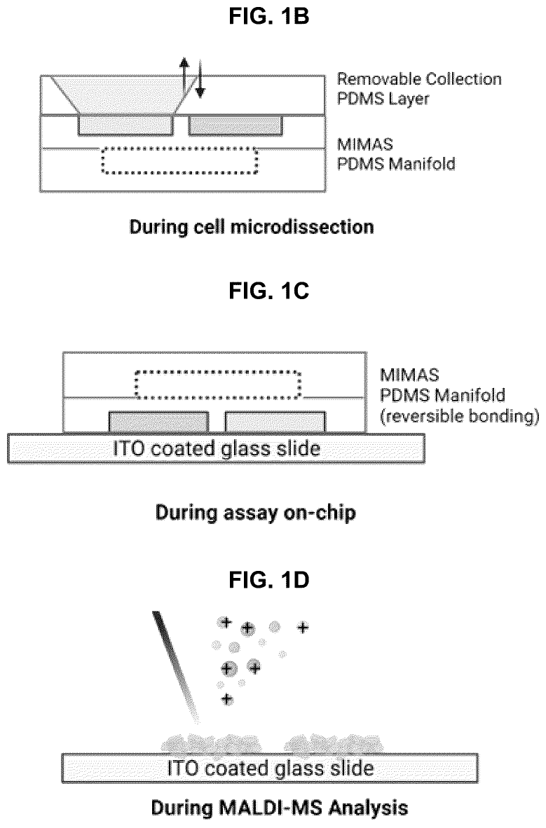

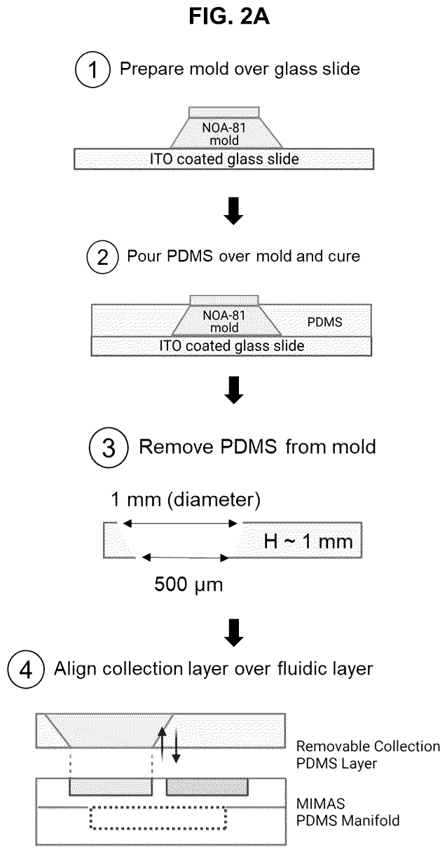

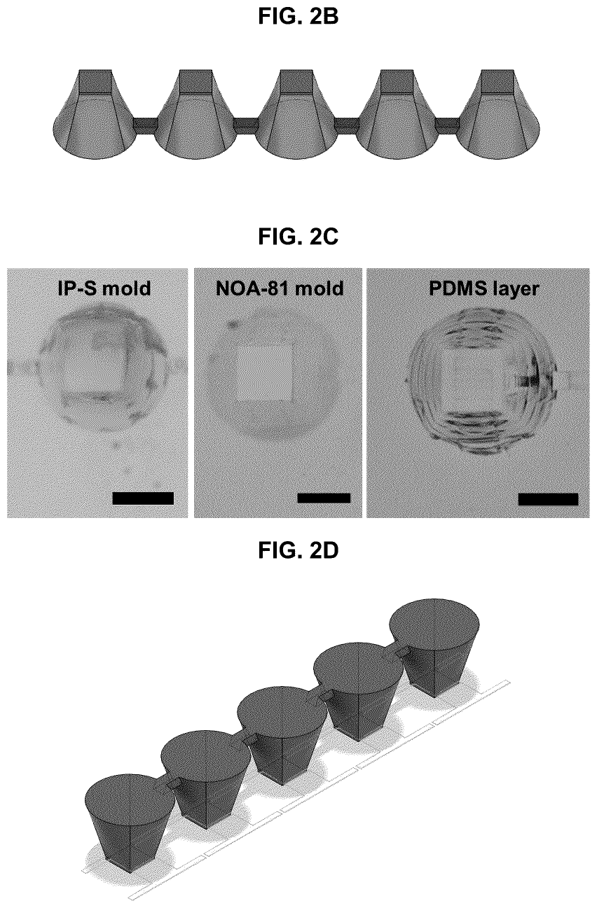

The MIMAS device (FIG. 1) consists of a double (fluidic and control) PDMS layer reversibly bonded to an ITO-coated glass slide. The channel height was 25 μm and 40 μm for the fluidic and the control layer, respectively. An additional layer (collection layer) was used with the original design to facilitate loading of the dissected cells into the MIMAS device (FIG. 2 and FIG. 1B). A thick PDMS slab with 2-mm diameter openings (milli-wells) on a glass slide was also employed for determination of the limit of detection (LOD).

### Collection Layer Fabrication

To load the cells into the MIMAS wells, a collection layer was designed and fabricated (shown in FIG. 2A). A mold (FIG. 2B) was designed using Fusion360 (Autodesk, USA). The mold was 3D-printed using a Nanoscribe Photonic Professional GT printer (Nanoscribe GmbH, Germany) and the Nanoscribe's proprietary photo-resin IP-S. The 3D-printed piece was developed in SU-8 developer and then UV-cured for 30 min as shown in FIG. 2C. This layer consists of 1 mm diameter circles funneled into a 500×500 μm square. An inverted mold of the 3D-printed piece was fabricated using PDMS (10:1 (w/w) ratio). The PDMS mold was then used to create a replica of the 3D-printed piece using NOA-81 epoxy and UV curing. The NOA-81 molds were used for the fabrication of the collection layer by pouring PDMS in a 10:1 (w/w) ratio of base to curing agent, degassing, and curing at 85° C. for 2 h. The PDMS collection layer was then cut to the MIMAS device dimensions, placed over it, and aligned by matching the 500 μm×500 μm openings to the MIMAS wells as schematically demonstrated in FIG. 2D. After the collection of microdissected cells, the collection layer was removed.

### Amyloid-β Monomer and Oligomer Solution Preparation

Aβ₁-42 and Aβ₁₋₄₀ monomers (Aβ-M) and oligomers (Aβ-O) were prepared as follows. Briefly, HFIP-treated Aβ peptide was aliquoted, lyophilized, and stored at −20° C. until used. For Aβ-M, the peptide was dissolved in 1 μL DMSO, sonicated for 10 min and diluted to 80 μM using ice-cold 10 mM sodium phosphate buffer. For Aβ-O, the peptide was dissolved in 5 μL DMSO, sonicated for 10 min, diluted to 150 μM using Ham's F-12 medium, and incubated at 4° C. for 24 h. After incubation, the solution was diluted to 80 μM using 10 mM sodium phosphate buffer. All further dilutions were performed with 10 mM sodium phosphate buffer. The composition of synthetic monomeric Aβ peptides was confirmed by MALDI-MS spotting the peptide solution mixed with matrix in a 1:1 ratio to the MALDI target plate, and by SDS-PAGE following standard procedures. For Aβ₁₋₄₂, the monomeric band was extracted from the gel and analyzed LC-MS/MS.

### Cell Laser Capture Microdissection and Collection

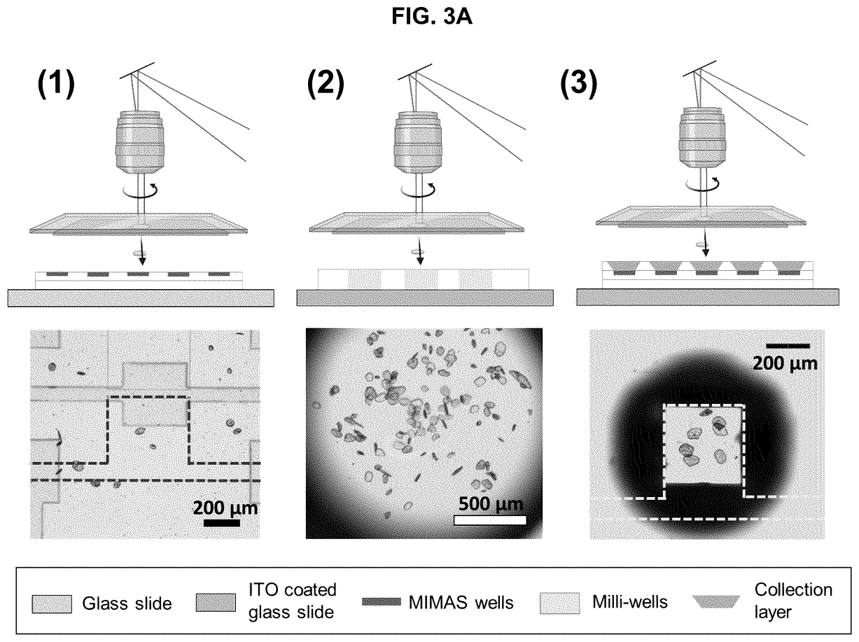

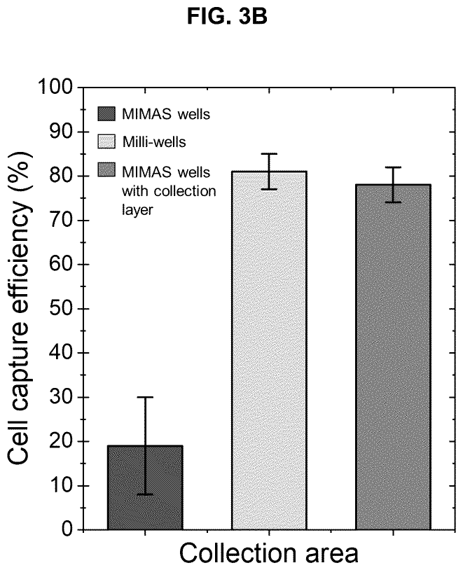

Frozen human tissue (brain slices from the middle frontal gyrus from non-AD tissue sections, without signs of amyloid plaques) was obtained from the Banner Sun Health Research Institute (Sun City, USA). A phase-contrast Leica LMD6500 microscope with a universal holder for collection devices (Leica Microsystems, Germany) was used for cell laser capture microdissection (LCM). Brain sections were inspected under the microscope to identify pyramidal cells based on their morphology. LCM 6.6 software (Leica Microsystems, Germany) was used to draw and cut over an outline around the body of each of the pyramidal cells to ensure the sample contained only intracellular contents. Once the laser cuts around the outline, the selected pyramidal cell's bodies fell into the collection area by gravity. The dissected cells were loaded directly into (a) a MIMAS device with its wells facing up (FIG. 3A), (b) milli-wells (FIG. 3B), or (c) a MIMAS device with a temporarily affixed collection layer, aligned over the respective wells (FIG. 3C). After dissection, the cells in each well were visually counted. Prior to cell collection, the MIMAS and milli-wells were filled with phosphate buffer. After collection, the buffer was allowed to evaporate (˜15 min) with the dissected cells in the wells. The collection layer, if present, was then removed, and an ITO-coated slide was assembled on the upwards-facing, cell-loaded MIMAS device (FIG. 1B). This assembly was turned upside down to carry out the fluidic steps of the assay with the glass side of the device forming the bottom substrate.

### In Vitro-Synthesized Amyloid-β Immunocapture

The immunocapture of Aβ was performed in milli-wells and MIMAS wells following an adapted, previously reported protocol for on-chip protein immunocapture and MS analysis. For the MIMAS platform, solution loading consists of valve opening by applying vacuum to the control layer inlets, allowing the solutions to fill up the wells by capillary action. The solutions were removed from the wells by opening the valves and applying a vacuum to the line outlets. For the MIMAS protocol, a solution of IgG 6E10 was loaded into the fluidic layer I by opening valve line A (FIG. 1). The IgG 6E10 antibody recognizes amino acids 1-16 of β-amyloid. The chip was incubated for 2 h at 36° C. in a humidity chamber. The wells were washed with 20 mM sodium phosphate buffer thrice and loaded with a 1% BSA in 10 mM sodium phosphate buffer blocking solution. The chip was incubated in a humidity chamber for 1 h at 36° C. followed by three washing steps, with 20 mM sodium phosphate buffer. Then, the Aβ solution was loaded into the wells, incubated for 1 h at room temperature (RT), and washed with 50 mM ammonium bicarbonate. Finally, the matrix solution was loaded into the fluidic wells II by controlling valve line B, and mixed with the immunocaptured Aβ by actuating valve line C. The solutions were then evaporated at RT overnight. Once the matrix was dry, the PDMS manifold was peeled-off, leaving only the co-crystallized matrix-analyte on the ITO-coated glass, which was then placed in the MALDI-MS instrument. All experiments were performed in triplicates (three MIMAS devices with 5 pairs of opposing wells each), and each well pair was analyzed individually. Aβ-M and Aβ-O were separately loaded, immunocaptured, and analyzed. The matrix used for Aβ-M was saturated a CHCA in 40% acetonitrile and 0.1% TFA, and for Aβ-O, 10 mg/mL sinapinic acid in 50% acetonitrile and 0.05% TFA.

Amyloid-β Immunocapture from Brain Cells

The developed immunoassay workflow was then carried out with brain cells. The brain cell protocol was established first with milli-wells and then with the MIMAS platform. The collection layer was aligned over the MIMAS device and filled up with buffer. The body of a pyramidal cell from non-AD brain tissue was identified by its unique morphology, micro-dissected, and allowed to fall into the collection layer opening. After the buffer in the well was dried, the collection layer was removed. An ITO-coated glass slide was cleaned with acetone and isopropanol and treated with oxygen plasma for 1 min under medium RF conditions. The PDMS MIMAS manifold containing the dissected cells was then bonded on a treated ITO-coated glass slide (FIG. 1C). Immunocapture in the assembled device was performed as described above. Briefly, IgG 6E10 was immobilized in fluidic line II and washed thrice with 20 mM sodium phosphate buffer. A 1% BSA blocking step was performed, followed by a triple 20 mM sodium phosphate buffer wash. The contents of opposing wells in fluidic lines I and II were mixed by actuating valve line C. The solubilized content of the dissected cells in line I with the immobilized IgG in fluidic line II were incubated for 1 hour at RT. Both fluidic lines were washed thrice with 50 mM ammonium bicarbonate. Finally, the matrix solution (10 mg/mL SA in 50% ACN and 0.05% TFA) was loaded into fluidic line I, mixed with the immunocaptured content in line II by opening the valve C line, and allowed to dry at RT overnight. The PDMS manifold was peeled-off, leaving the exposed crystals on the ITO-coated glass slide, which was then used as the target in the MALDI-MS instrument (FIG. 1D).

### MALDI Mass Spectrometry Analysis

MALDI-MS analysis was performed by placing the sample on the ITO-coated glass target in a Bruker Microflex LRF in linear mode. Crystals were identified on the ITO slide by visual inspection using the instrument's camera. The LOD of Aβ was determined with monomeric Aβ₁₋₄₀ and Aβ₁₋₄₂ at various concentrations, pre-mixed with matrix, loaded into the milli-wells and the MIMAS wells, and allowed to dry at RT. For MS analysis using milli-wells (n=3), 4,000 shots/milli-well were averaged. For MS analysis using the MIMAS device (3 devices=15 MIMAS wells), the crystals in a well were depleted and averaged (>2,000 shots/well). The LOD was determined using Origin (OriginLab, USA) by linear fitting the standard curve and LOD=(3.3 σ)/S; where σ is the y-intercept standard deviation and S the slope. The reported m/z values correspond to the average mass in the MS spectrum.

### In Vitro-Synthesized Aβ Immunocapture Using Milli-Wells

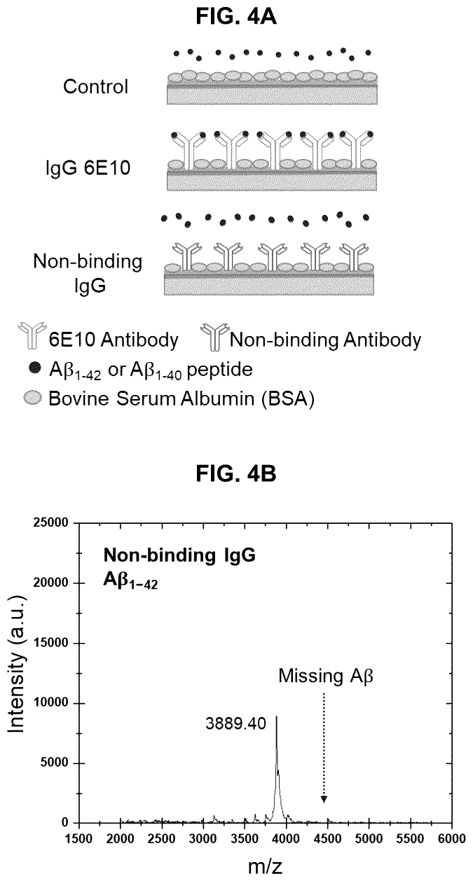

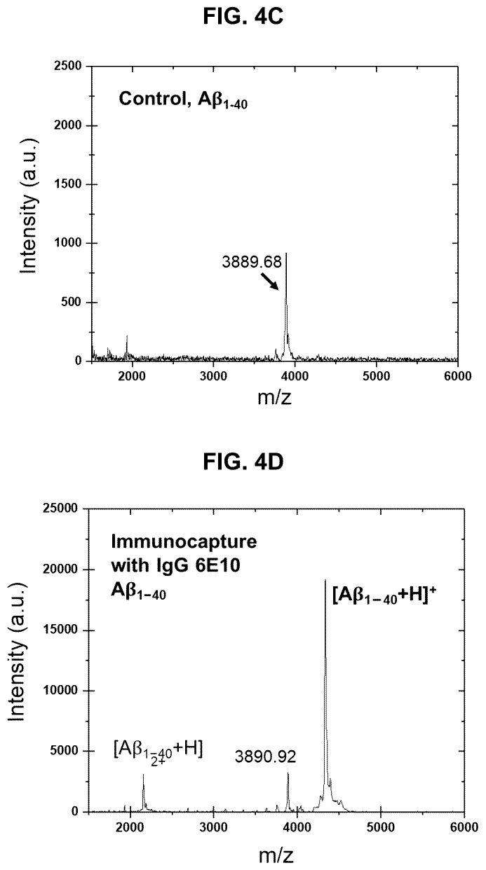

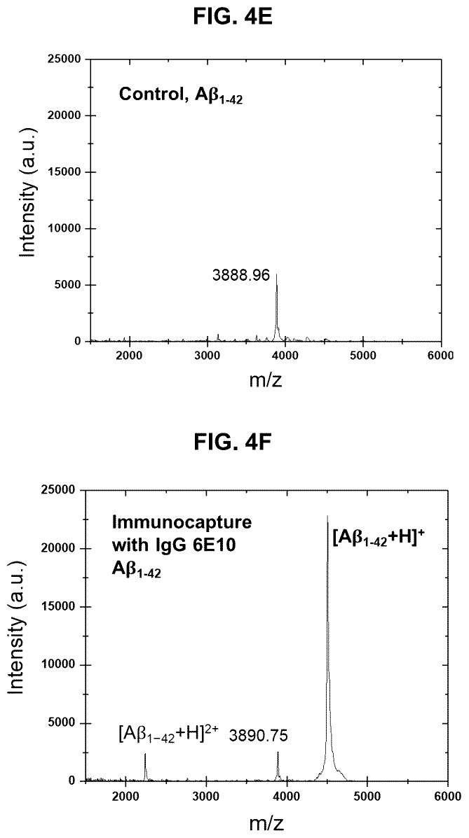

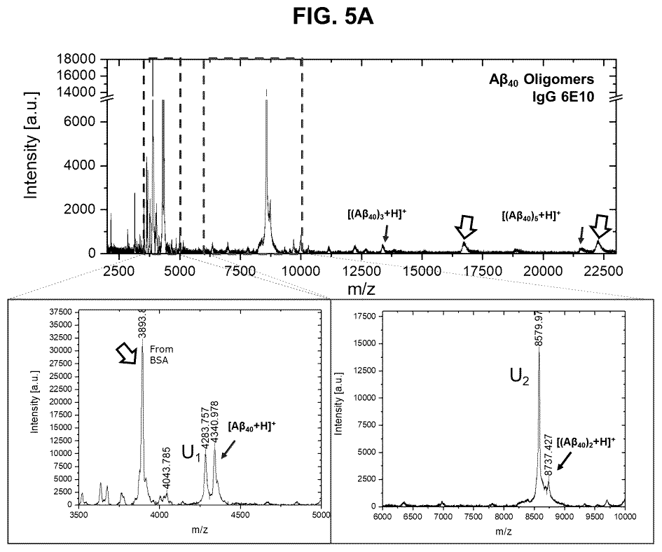

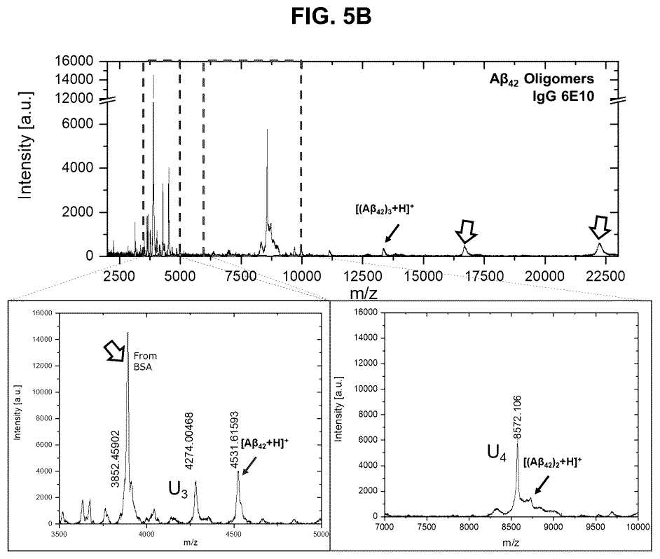

The immunocapture of Aβ was performed in milli-wells mounted on ITO-coated glass slides. First, 5 μL of 0.05 mg/mL immunoglobulin G (IgG) 6E10 in 10 mM phosphate buffer were loaded into each milli-well and incubated for 2 h at 36° C. in a humidity chamber. Then, milli-wells were washed with 20 mM sodium phosphate buffer three times. A blocking step was performed by loading 5 μL of 1% BSA in 10 mM phosphate buffer to each milli-well and incubating in a humidity chamber for 1 h at 36° C., followed by washing with 20 mM sodium phosphate buffer thrice. Then, 5 μL of Aβ solution was loaded into the milli-wells and incubated for 1 h at room temperature (RT), followed by a wash step with 50 mM ammonium bicarbonate which was repeated thrice. Finally, the matrix solution was loaded into the milli-wells and the contents dried at RT for co-crystallization. Once dried, the PDMS manifold was peeled-off from the glass slide. The ITO-coated glass slide was then used as the target in the MALDI-MS instrument. All experiments were performed in triplicates (three milli-wells per condition). A saturated solution of α-CHCA matrix in 40% acetonitrile and 0.1% TFA was used for Aβ-M. A 10 mg/mL solution of sinapinic acid in 50% acetonitrile and 0.05% TFA was used as the matrix for Aβ-O. Representative spectra of the Aβ-M and Aβ-O immunocapture using milli-wells are shown in FIG. 4 and FIG. 5, respectively.

Amyloid-β Immunocapture from Brain Cells Using Milli-Wells

First, 5 μL of 0.05 mg/mL IgG 6E10 solution was loaded into the reservoir and incubated for 2 h at 36° C. in a humidity chamber. The reservoirs were washed three times using 20 mM sodium phosphate buffer. Next, 5 μL of 1% BSA blocking solution was loaded into the reservoir and incubated 1 h at 36° C., followed by a washing step with 20 mM sodium phosphate buffer. Then, the reservoirs were placed on the LCM instrument for brain cell dissection and loaded as described in the cell microdissection and collection section above. Then, the devices were incubated for 1 h at RT, followed by a washing step with 50 mM ammonium bicarbonate. Finally, 5 μL of matrix solution was added to the reservoirs. Once the solution was dried, the PDMS manifold was peeled-off from the glass slide, and the slide was then used as the target in the MALDI-MS instrument. All experiments were performed in triplicates. A 10 mg/mL matrix solution of sinapinic acid in 50% acetonitrile and 0.05% TFA was used for experiments with brain cells.

### Example 2

Developing the current assay for Aβ from brain cells entailed several important steps: optimization of cell transfer into the microfluidic platform, MS detection limit characterization, immunocapture, characterization of bound Aβ species with MALDI MS, and workflow implementation in the MIMAS platform. Along with the integration of all the functional microfluidic elements necessary for an entirely on-chip assay (FIG. 1), a multi-level protocol for efficient transfer of laser-dissected cells into the platform wells (fluidic line I, FIG. 1), antibody functionalization, Aβ immunocapture, matrix co-crystallization, and MALDI MS analysis were developed. Incubation and washing steps were implemented with integrated membrane valves and additional wells (fluidic line II, FIG. 1). The protocol ends with the removal of the multi-layered microfluidic elastomeric manifold within which the sample has been processed in nanoliter-chambers formed by the MIMAS wells. The sample remains on the conductive glass slide, ready for in-situ MALDI MS analysis.

### Brain Tissue Cell Microdissection and Collection

Collection directly into the MIMAS device wells implies gravity translocation of the cells to a 0.25 mm² area, more than 60 times smaller than the collection area presented by the 4.5 mm-diameter microtube caps typically used with the LCM. The LCM capture efficiency of cells from tissue into such caps is around 90%; however, it can be as low as 20% to 50% for low humidity conditions or tissue sections with dimensions larger than a hundred micrometers. To study the transfer efficiency of this platform, three methods were tested, which are schematically represented in FIG. 2: (i) collection directly into MIMAS device wells (FIG. 3A); (ii) collection into 2 mm-diameter milli-wells (FIG. 3B); and (iii) collection into the MIMAS device with the aid of a collection layer fabricated in PDMS elastomer, which provides a 1 mm diameter opening to funnel the cells into the MIMAS wells (FIG. 3C). The resulting capture efficiencies are summarized in FIG. 3D. As expected, due to the small collection area, the MIMAS wells exhibited the lowest, 19±11% collection efficiency. The 2 mm-diameter milli-wells capture efficiency was 81±4%. The capture on the MIMAS device with the 1 mm-diameter collection layer funnels resulted in a 78% efficiency. Thus, the collection layer increases translocation efficiency into the MIMAS wells from 19±11% to 78±4%. Conveniently, any debris or cells not translocated to the wells are removed with the collection layer. This is an advantage because any misplaced dissected cells on device surfaces other than the MIMAS-wells could interfere with subsequent assembly steps (see example in FIG. 3A). Therefore, all further experiments with the MIMAS devices were performed using the removable collection layer. The 78% collection efficiency of the MIMAS devices with the collection layer is comparable to that of standard 0.2 to 0.5 ml tube caps; however, the volume of a MIMAS well is a few nanoliters. Compared with standard sample preparation in microtubes, LCM-MIMAS provides a reduction of sample dilution by several orders of magnitude.

### Amyloid-β Immunoassay On-Chip

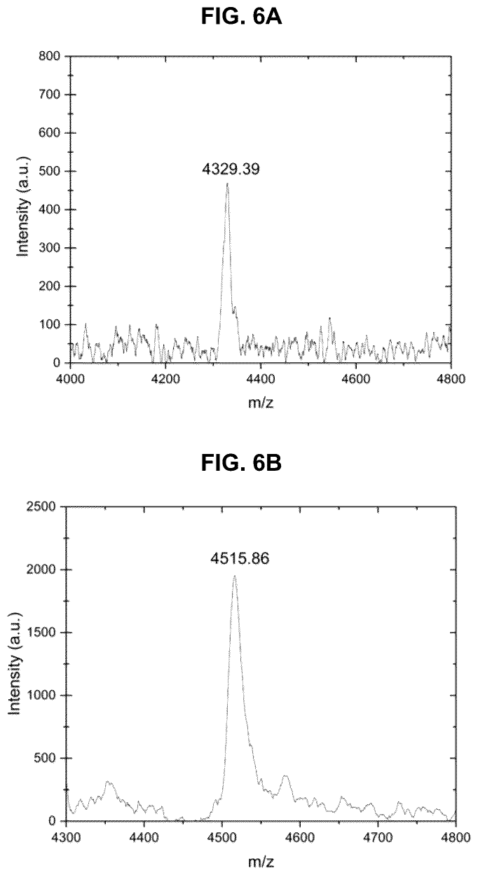

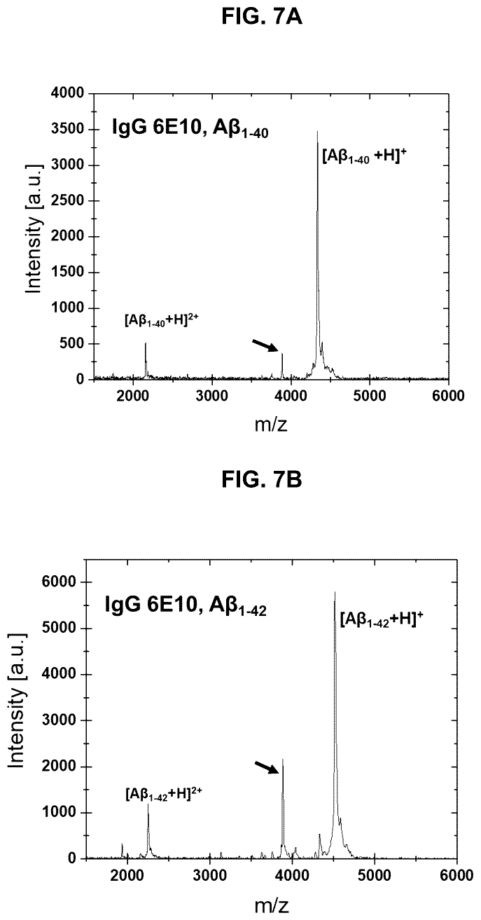

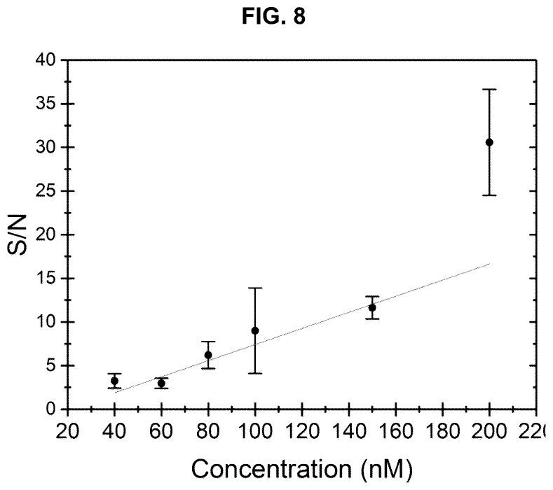

The immunocapture assay was first developed and optimized in milli-wells using IgG 6E10, then developed in the MIMAS platform. The selected IgG is one of the most commonly used for AD research, known to bind both monomeric and oligomeric Aβ species. The IgG 6E10 binding epitope corresponds to the human Aβ amino acid residues 5 to 7. The affinity of IgG 6E10 for monomeric and oligomeric Aβ₁₋₄₀ and Aβ₁₋₄₂ was verified using synthetic Aβ solutions in both the milli-wells and the MIMAS platform. For Aβ₁₋₄₀-M and Aβ₁₋₄₂-M solutions incubated with immobilized IgG 6E10, MS spectra peaks [Aβ+H]⁺ and [Aβ+2H]²⁺ confirmed the affinity binding of the monomer peptides to the IgG (FIG. 6A-B). The absence of the peaks in the control experiments without an antibody and using a non-binding antibody (IgG A11 specific for Aβ-O, which does not bind Aβ-M) confirmed minimal non-specific binding. A peak with m/z ˜3880 was observed in all cases, which, as previously reported, is associated with the BSA blocking step. The spectra from the Aβ₁₋₄₀-M and Aβ₁₋₄₂-M immunoassay in the MIMAS platform are shown in FIG. 7A-B. As in the milli-well immunoassay, peaks corresponding to [Aβ+H]⁺ and [Aβ+2H]²⁺ were observed for Aβ₁₋₄₀-M and Aβ₁₋₄₂-M solutions exposed to IgG 6E10, and absent in the controls, confirming the lack of non-specific binding (FIG. 5). In this assay, the LOD of the immunocaptured Aβ₁₋₄₂ was estimated to be 64.2 nM (with a S/N>3), equivalent to approximately 3.38×10⁸ molecules per MIMAS well (see FIG. 8 for the standard curve).

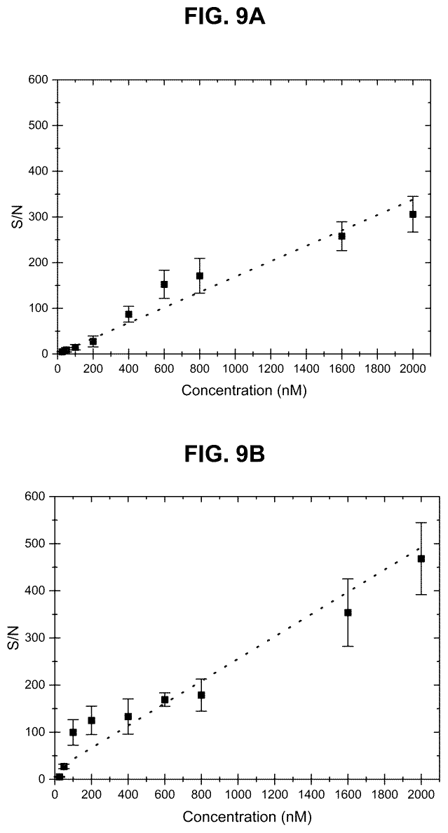

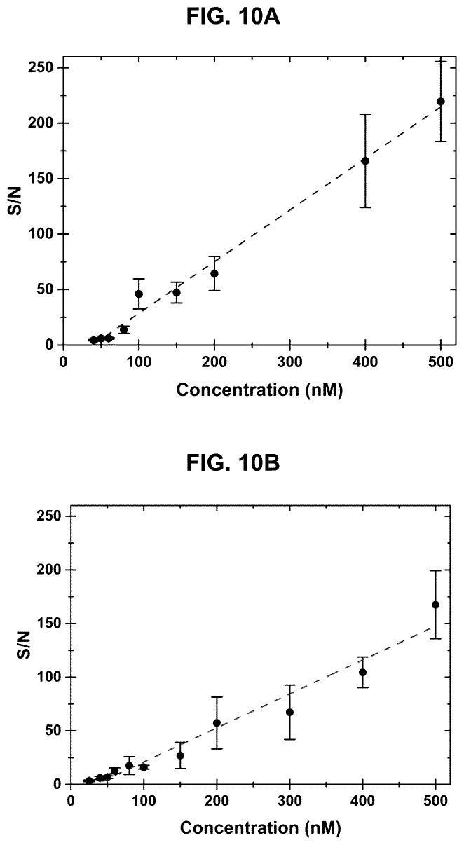

It is worth comparing this assay based Aβ₁₋₄₂ LOD with a standard curve for various concentrations of Aβ-M (see FIG. 9 for Aβ₁₋₄₀ and FIG. 10 for Aβ,_42). The LOD using the milli-wells was estimated as 80.64 nM and 96.13 nM for Aβ₁₋₄₀ and Aβ₁₋₄₂, respectively. Considering the 5 μL volume of milli-wells, the LOD is equivalent to 2.42×10¹¹ molecules of Aβ₁₋₄₀ and 2.89×10¹¹ molecules of Aβ₁₋₄₂. For the MIMAS platform, the LOD of Aβ₁₋₄₀ and Aβ₁₋₄₂ was calculated as 31.2 nM and 51.17 nM, respectively. Thus, the LOD for Aβ₁₋₄₂ in the MIMAS wells, including the entire immunocapture procedure (see above), is only slightly higher than in the experiments performed filling up wells with various concentrations of Aβ. In addition, with a well volume of 8.75 nL, an equivalent of 1.64×10⁸ molecules of Aβ₁₋₄₀ and 2.69×10⁸ molecules of Aβ₁₋₄₂ per well is calculated. Furthermore, a representative spectrum of both peptides close to the respective LOD is shown in FIG. 10.

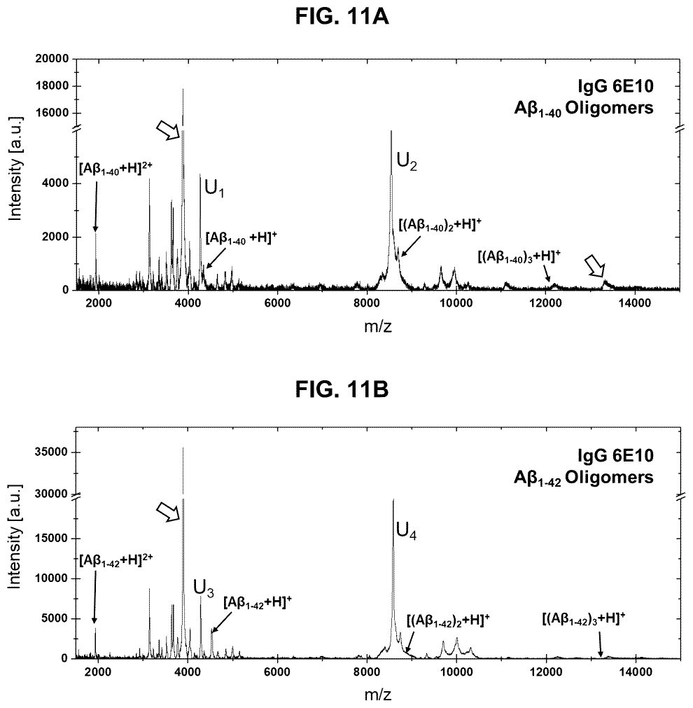

In vitro synthesized Aβ₁₋₄₀-O and Aβ₁₋₄₂-O were used to assess oligomer IgG 6E10 capture and bound species by MS. Oligomeric species of Aβ₁₋₄₀ up to 9-mers and Aβ₁₋₄₂ up to 12-mers have been detected using MALDI-MS, which indicates MALDI-MS can be used to characterize Aβ-O IgG 6E10 capture. The spectra of immunocaptured Aβ₁₋₄₀-O and Aβ₁₋₄₂-O in milli-wells are included in FIG. 4, while the spectra using the MIMAS platform are shown in FIG. 11. For Aβ₁₋₄₀-O, monomers (m/z 4,335, [Aβ₁₋₄₀+H]⁺), dimers (m/z 8,579, [(Aβ₁₋₄₀)₂+H]⁺) and trimers (m/z 12,993, [(Aβ₁₋₄₀)₃+H]⁺) were observed. Similarly, for Aβ₁₋₄₂-O, monomers (m/z 4,517, [Aβ₁₋₄₂+H]⁺), dimers (m/z 9,070, [(Aβ₁₋₄₂)₂+H]⁺), and trimers (m/z 13,482, [(Aβ₁₋₄₂)₃+H]⁺) were observed. Interestingly, high intensity peaks at ˜m/z 4,270 and m/z ˜8,580 were observed for both Aβ₁₋₄₀-O and Aβ₁₋₄₂-O. These peaks do not match any expected m/z for oligomeric species; thus, for further discussion, these peaks are referred to as U1 and U2 for Aβ₁₋₄₀, and U3 and U4 for Aβ₁₋₄₂. All peaks observed in the MS analysis of Aβ₁₋₄₀-O and Aβ₁₋₄₂-O are summarized in Table 1 and Table 2, respectively. Previously, Anker et al., reported the assessment of synthetic Aβ-O with MALDI-MS identifying Aβ₁₋₄₂ monomers at m/z 4,520, dimers at m/z 9,051, and trimers at m/z 13,590 (while other oligomers were detected, the specific m/z was not reported), amounting in the same mass difference as observed in our work for the monomer. Anker et al., J. Phys. Chem. C 113 (15): 5891-5894 (2009). In contrast, the mass difference for the dimer observed here was a factor two larger (see Table 2). Similar to our work, with higher mass ranges, the intensity of the signal decreases in agreement with the expected decrease in sensitivity for MALDI MS as the molecular weight of the assessed molecule increases as reported by Anker et al., J. Phys. Chem. C 113 (15): 5891-5894 (2009) and Wang et al., J. Am. Soc. Mass Spectr. 29 (4): 786-795 (2018).

TABLE 1
Resulting peaks from Aβ₁₋₄₀-O
immunoassay and MALDI-MS analysis

| Peak Assignment | Detected m/z | Δ m/z | Calculated mass (Da) |
|---|---|---|---|
| U1 | 4,270.6 | n/a | — |
| [Aβ₁₋₄₀ + H]⁺ | 4,335.5 | 5.5 | 4,330 |
| U2 | 8,579.5 | n/a | — |
| [(Aβ₁₋₄₀)₂ + H]⁺ | 8,658.3 | 8.3 | 8,660 |
| [(Aβ₁₋₄₀)₃ + H]⁺ | 12,993.5 | 3.5 | 12,990 |

TABLE 2
Resulting peaks from Aβ₁₋₄₂-O
immunoassay and MALDI-MS analysis

| Peak Assignment | Detected m/z | Δ m/z | Calculated mass (Da) |
|---|---|---|---|
| U3 | 4,284.9 | n/a | — |
| [Aβ₁₋₄₂ + H]⁺ | 4,517.5 | 3.5 | 4,514 |
| U4 | 8,581.6 | n/a | — |
| [(Aβ₁₋₄₂)₂ + H]⁺ | 9,070.2 | 42.1 | 9,028 |
| [(Aβ₁₋₄₂)₃ + H]⁺ | 13,482.1 | 60.1 | 13,542 |

The IgG 6E10 affinity for Aβ monomers, oligomers, and fibrils has been well established, although without much detail on specific low molecular weight oligomeric species binding. Aβ monomers, dimers, and trimers have been found in human brain homogenate immunoblotting. Pham et al. reported the detection of up to heptamers using IgG 6E10 in brain homogenate immunoblots, with the caveat that the trimers and tetramers were not fully resolved (Pham et al., FEBS J. 277 (14): 3051-3067 (2010)). The immunoassay with MS analysis demonstrated here provides a distinctive advantage to further assess the 6E10 antibody binding to lower molecular weight oligomers. To our knowledge, there are no previous reports of in-vitro MS characterization of Aβ-O species immunocaptured with IgG 6E10. In this work, Aβ₁₋₄₀-O up to the trimeric species were identified by MALDI-MS after on-chip immunocapture. This indicates that MS analysis allows sensitive identification of distinct immunocaptured oligomeric and other species that might not be identified by previously employed methods.

There are several possible reasons for larger oligomers not being found after the immunoassay, although in vitro-generated species can be detected by MALDI MS. Large oligomer binding by the 6E10 antibody might be inhibited. It is also possible that these species are either transient or in concentrations too low for MS. It also seems plausible that larger oligomers might dissociate into smaller species during MS. However, oligomers up to 12-mers were identified in in vitro preparations with MALDI-MS in preliminary work, and have also been reported by Wang et al., J. Am. Soc. Mass Spectr. 29 (4): 786-795 (2018). The peaks corresponding to the monomers and dimers exhibit a wide m/z distribution, which could originate from the dissociation of larger species or multi-charged larger species that cannot be resolved with the employed MALDI-MS instrument.

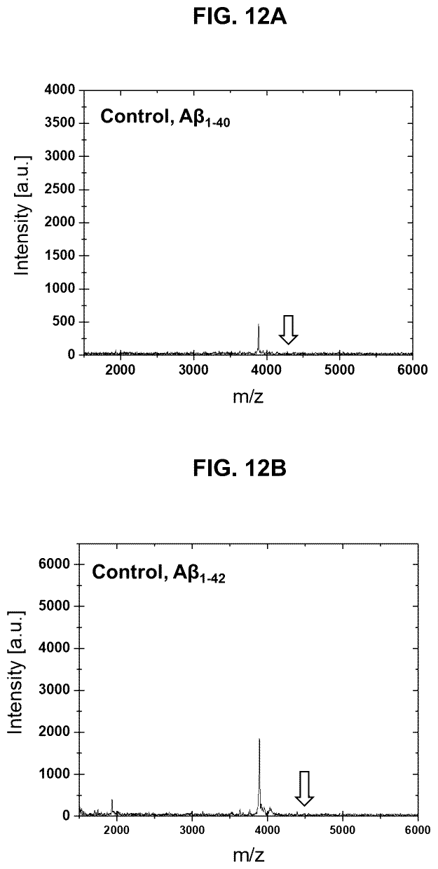

Control experiments without IgG were also performed to test for non-specific oligomer binding. In addition to the peak with m/z ˜3880 associated with the BSA blocking step often observed in the MIMAS assays, peaks at m/z ˜66,000, ˜33,000, ˜22,000, and ˜16,500 were also observed (see the representative spectrum in FIG. 12). The m/z 3880 peak related to the blocking step has been observed through the development of this microfluidic approach, typically performed using a saturated α-HCCA solution as the matrix (peptides <5 kDa were typically assessed). It is known that matrix selection and optimization are critical for assay development and can significantly impact analyte ionization. Here, a sinapinic acid matrix was used for Aβ-O assays (recommended for >10 kDa molecules), which may explain the appearance of peaks that can be related to single and multiple-charged species as well as to the intact BSA molecule (MW 66 kDa) used in the blocking step. To avoid the presence of these peaks, either optimization of the matrix solution or of BSA concentration (in the blocking step) can be performed.

It is interesting to note that the peaks not associated with oligomeric species that appear in the Aβ₁₋₄₀-O spectra (U1/U2) and in the Aβ₁₋₄₂-O spectra (U3/U4) have a lower m/z than expected monomeric or dimeric species (see Tables 1 and 2). The higher m/z peaks U2 and U4 may be considered a dimer version of U1 and U3, respectively. Alternatively, these peaks may also represent multi-charged species of larger oligomers. It is important to mention that MALDI MS was performed in linear mode, as otherwise (i.e., using the reflectron mode), the sensitivity to detect the Aβ-O was not reached. Additionally, the limited resolution of the instrument did not allow for identification of the observed peaks and further work to properly define the unknown species U1-U4 must be performed.

Analysis of Amyloid-β from Brain Cells in Milli-Wells

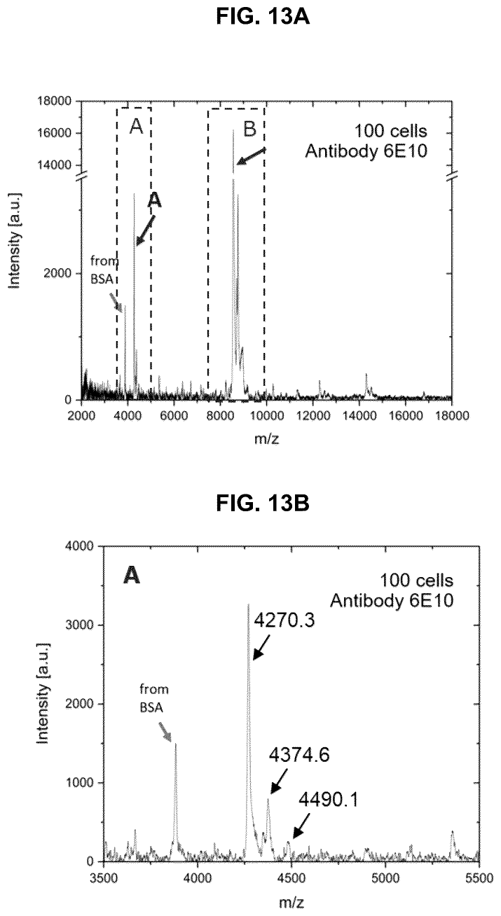

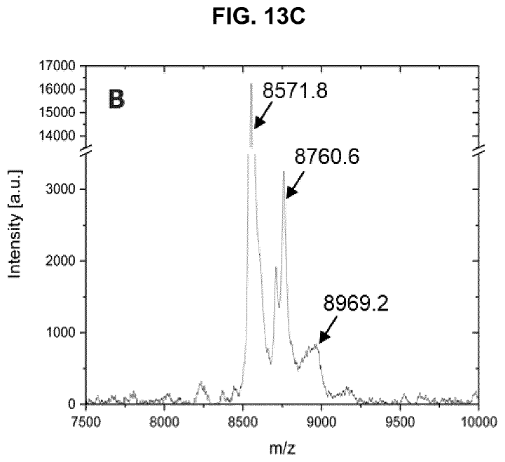

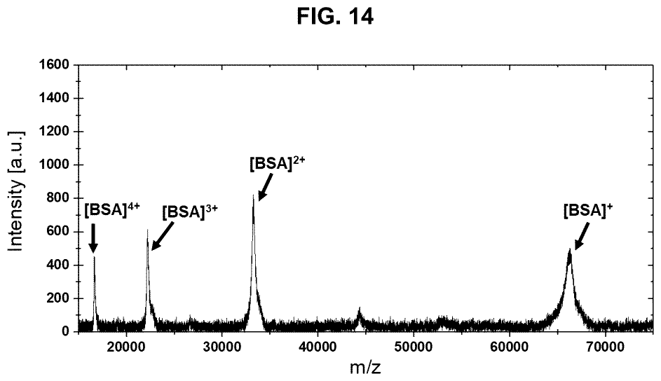

To demonstrate Aβ extraction and immunocapture from microdissected brain tissue cells, the immunoassay was first performed with brain cells in milli-wells. Pyramidal cells from frozen brain tissue sections were identified based on their morphology. The body of pyramidal cells was selected for dissection to avoid the use of extracellular material in the assay. Experiments were performed in milli-wells filled with 20 mM phosphate buffer and 100 microdissected cells per well. As a control, 100 cells were collected on wells without immobilized antibodies. A representative spectrum obtained after extraction and immunocapture is shown in FIG. 13A. The peaks observed when the IgG is present (FIG. 13A, marked with black arrows) are missing in the controls without immobilized IgG (see FIG. 14 for a representative control MS spectrum). FIG. 13B shows a group of zoomed-in peaks below 5,500 m/z: m/z 4,270, m/z 4,374, and m/z 4,490. FIG. 13C shows peaks higher than 7,500 m/z (m/z 8,570, 8,760.6, and 8,970). Interestingly, a pattern similarity was found between the peak groups. Based on this observation, the first group of peaks could be related to Aβ monomeric species, while the second could correspond to the dimeric forms of the first group.

Furthermore, the peak assignment of expected Aβ species was compared with those obtained by MALDI-MS and other potential candidates (Table 3). Aβ species other than Aβ₁₋₄₀ in brain homogenate have been identified by immunoprecipitation (IP) with the 6E10 antibody and MALDI-MS. The m/z 4270.3 peak was assigned to Aβ₃₉ with either a K⁺ adduct ([Aβ₃₉+K]⁺) or an acetonitrile adduct ([Aβ₃₉+ACN+H]⁺). Potential adducts were considered based on common adducts present in MALDI-MS based on the Mass Spectrometry Adduct Calculator and are listed in Table 3 together with calculated masses. Since Aβ₃₉ has been identified by analysis of bulk brain samples using IgG 6E10 via IP and MALDI-MS, 20, 62 and K⁺ is present in the cell lysis buffer while acetonitrile is present in the matrix solution, the two potential adducts seem likely. Correspondingly, the m/z of 8571.8 could be the Aβ₃₉ dimer either as a K⁺ or acetonitrile adduct. Similarly, the m/z 4374.6 and m/z 8760.6 peaks match the m/z of Aβ₁₋₄₀ monomers and dimers with potential adducts of Na⁺, K⁺, or acetonitrile. Lastly, m/z 4490.1 and m/z 8969.2 peaks can be matched to the Aβ₂₋₄₃ monomer and dimer, respectively. Interestingly, peaks identified as [Aβ₁₋₃₉+K]⁺ or [Aβ₁₋₃₉+ACN+H]⁺ are close in m/z to the U1 and U2 peaks observed in the immunocapture of Aβ-O (see FIG. 11 and FIG. 4). In addition, the mass differences are below 5 Da for the monomer species and below 32 Da for the dimer species, whereas the latter is attributed to the limited resolution of the employed Microflex instrument.

TABLE 3
Peaks detected from brain cells in milli-wells (all peaks)

| Observed | | Calculated mass for | |
|---|---|---|---|
| m/z | Potential Aβ species | Aβ species adducts | Δ m/z |
| 4270.3 | [Aβ₁₋₃₉ + K]⁺ | 4,270.0 | 0.3 |
| | [Aβ₁₋₃₉ + ACN + H]⁺ | 4,273.0 | 3.0 |
| *4270.2 | [Aβ₁₋₃₉ + K]⁺ | 4,270.0 | 0.2* |
| | [Aβ₁₋₃₉ + ACN + H]⁺ | 4,273.0 | 2.9* |
| | [Aβ₁₋₄₀ + 2Na − H]⁺ | 4,375.0 | 0.4 |
| 4374.6 | [Aβ₁₋₄₀ + K]⁺ | 4,369.0 | 5.6 |
| | [Aβ₁₋₄₀ + ACN + H]⁺ | 4,372.0 | 2.0 |
| 4490.1 | [Aβ₂₋₄₃ + H]⁺ | 4,500.8 | 9.9 |
| 8571.8 | [(Aβ₁₋₃₉)₂ + K]⁺ | 8,539.9 | 31.9 |
| | [(Aβ₁₋₃₉)₂ + K]⁺ | 8,546.1 | 25.7 |
| *8557.9 | [(Aβ₁₋₃₉)₂ + ACN + H]⁺ | 8,539.9 | 18* |
| | [(Aβ₁₋₃₉)₂ + ACN + H]⁺ | 8,546.1 | 11.8* |
| | [(Aβ₁₋₄₀)₂ + 2Na − H]⁺ | 8,749.9 | 10.7 |
| 8760.6 | [(Aβ₁₋₄₀)₂ + K]⁺ | 8,737.9 | 22.7 |
| | [(Aβ₁₋₄₀)₂ + ACN + H]⁺ | 8,744.1 | 16.5 |
| 8969.2 | [(Aβ₂₋₄₃)₂ + H]⁺ | 9,000.0 | 30.8 |

*Species marked with asterisks were observed in the spectra from brain cells assessed with the MIMAS device.

Analysis of Amyloid-β from Brain Cells in the LCM-MIMAS Platform

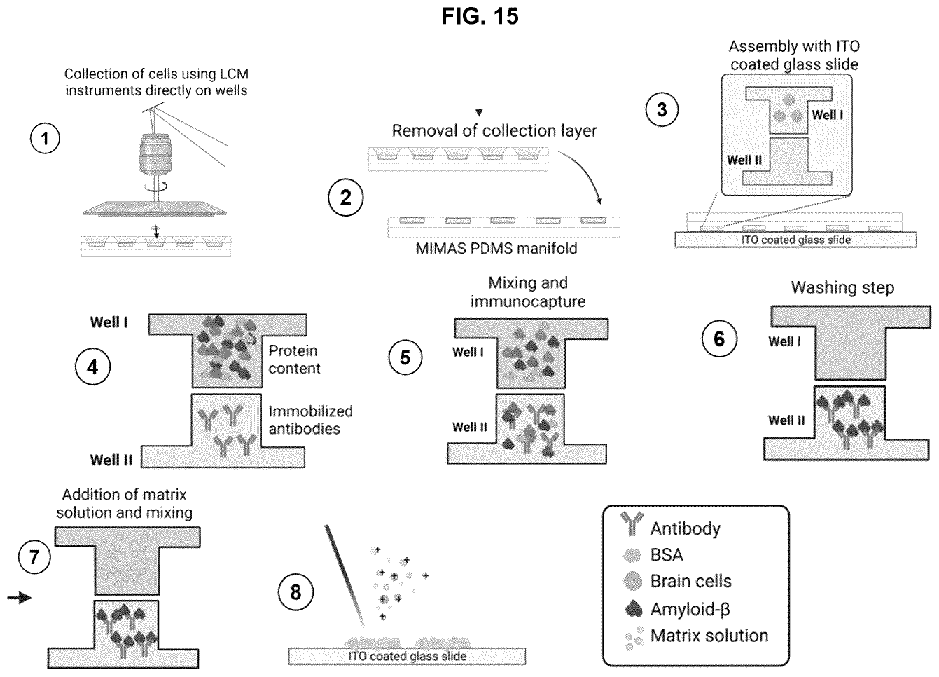

The LCM-MIMAS workflow (FIG. 15) was performed to assess the intracellular Aβ species in a small population of archived-brain laser-microdissected cells. The MIMAS manifold with phosphate buffer-filled wells was placed on a glass slide in the LCM instrument universal holder (see FIG. 3C). Cells were dissected into the MIMAS wells using the collection layer. After the collection layer removal, the MIMAS manifold with the cells was assembled onto an ITO-coated glass slide and incubated to extract protein content from the dissected cells while IgG 6E10 was immobilized in the well line II. The protein extract was delivered to the well with immobilized IgG by valve actuation, and Aβ immunocapture took place. After matrix addition and co-crystallization, the PDMS manifold was removed, leaving the co-crystals originating from the MIMAS wells on the ITO slide portion of the device, and MALDI MS analysis was performed.

The minimum number of cells for the LCM-MIMAS approach was estimated based on reported Aβ content in neurons and reservoir size and shape limitations. Soluble Aβ ranges from about 1 to 104 pg per μg of total neuron protein content. According to this, a 15 μm-thick neuron slice can be expected to contain 1.5×10⁴ to 1.5×10⁸ Aβ molecules. The LOD in a MIMAS well (FIG. 9) is close to the upper limit of the reported Aβ molecules per cell. On the other hand, the maximum MIMAS well loading capacity is ˜60 cells, based on the volume of an average pyramidal cell from a 15 μm-thick tissue section. Therefore, 20 cells were used to perform the MIMAS assay.

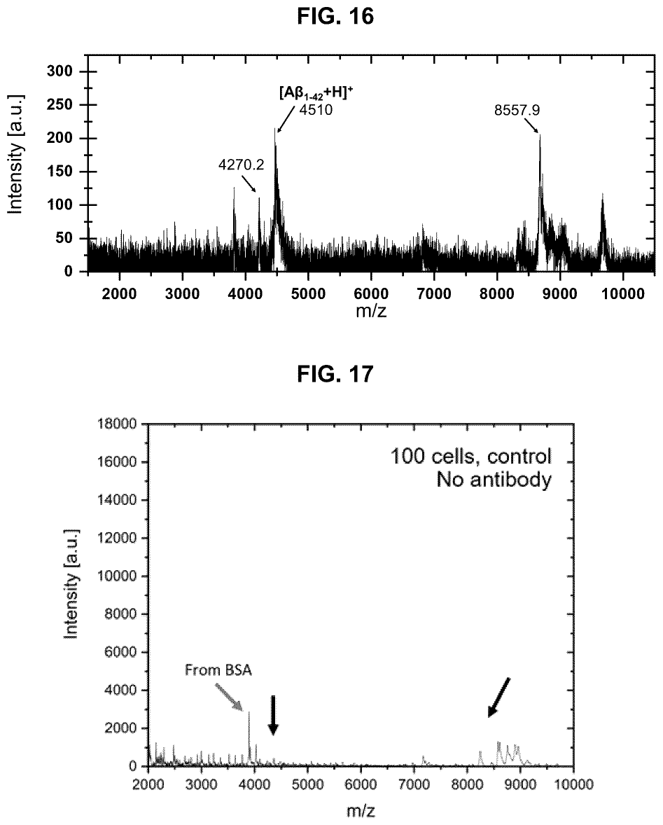

Using the MIMAS assay, peaks from the brain cells were successfully identified (representative spectrum shown in FIG. 16). The assay was performed in three MIMAS devices with five wells per device (n=15), and 20 cells per well. Peaks similar to those obtained with the milli-wells with m/z 4270.2 (±5 Da) and m/z 8557.9 (±7 Da) were observed in 10 and 11 wells of the 15 assessed MIMAS wells (FIG. 13). Peak m/z 4270.2 was matched to two potential candidates (see Table 3): [Aβ₃₉+K]⁺ (with an m/z calculated of 4,269.96) or [Aβ₃₉+ACN+H]⁺ (with an m/z calculated of 4,273.03). Peak m/z 8557.9 is likely a dimeric form of the m/z 4270.2 species. Furthermore, a peak observed around m/z 4,510.9 (±6 Da, observed in 5 of the 15 wells) was assigned to Aβ₁₋₄₂, which was not apparent in the milli-well assay performed with 100 cells. A variation in the type of species detected in different wells could be due to cell heterogeneity. To confirm the specificity of the assay, control experiments were also performed without IgG to rule out non-specific binding, similar to those for milli-wells (see FIG. 17). The peak assignment provided in this work constitutes a hypothesis based on the assay conditions and literature using similar conditions and the 6E10 antibody, which has been widely used for Aβ studies. Due to the limited resolution of the MS instrument and lack of MS/MS capabilities, a definite assignment of the observed Aβ species will be subject to future work, including studies on AD brain tissue samples. However, reported here is the capability of the LCM-MIMAS platform to perform an immunoassay from intracellular material of brain tissue and assessment of a specific peptide and its oligomeric species using MALDI-MS from a small number of brain slices.

The extremely high sensitivity of the LCM-MIMAS assay for Aβ-species from as few as 20 cells is similar to our previously demonstrated MIMAS assay with MCF-7 breast cancer cells, where the apoptosis-related protein Bcl-2 was detected from as few as 10 cells. This extremely high sensitivity is attributed to a reduced cell lysate complexity that limits analyte masking as well as to the overall MIMAS workflow that minimizes sample loss and dilution effects.

This work demonstrates the LCM-MIMAS platform for the analysis of Aβ species in small cell populations from archived brain tissue. A workflow was developed entirely on a chip, starting from laser-microdissection of cells into a microfluidic platform and ending in MALDI MS identification of IgG 6E10 immunocaptured Aβ-M and Aβ-O species from healthy human brain tissue. A novel capture element for coupling LCM with the MIMAS platform has been demonstrated, allowing the collection of exclusively intracellular components in the LCM-MIMAS assay. In the milli-wells, detection of immunocaptured intracellular Aβ species was achieved with ˜100 dissected cells from archived brain tissue. In the MIMAS platform, Aβ species were identified with as few as 20 cells. Monomer and dimer adduct candidate species of Aβ₁₋₃₉, Aβ₁₋₄₀, and Aβ₂₋₄₃ have been identified in the brain cell mass spectra when the assay was performed in the milli-wells and the MIMAS platform. However, in the MIMAS platform, an additional species was identified as Aβ₁₋₄₂. The in situ identification of Aβ species from as few as 20 cells from archived brain tissue sections puts forward the LCM-MIMAS approach as a powerful tool to elucidate intracellular Aβ species further. The LCM-MIMAS approach is advantageous compared to in vitro and in vivo Aβ-O characterization through commonly used gel electrophoresis because sodium dodecyl sulfate (SDS) in gels compromises the oligomer structural integrity, as reported by multiple studies. Although alternatives to avoid SDS exist, gel electrophoresis lacks the appropriate resolution to identify individual oligomers. LCM-MIMAS is suitable for studying small cell subpopulations with a defined type and disease state as well as extracellular species, towards elucidating the origin of AD and other diseases through a novel, sensitive assay for crucial disease proteins.

What is claimed:

1. A method for analyzing tissue for the presence of Aβ-M and Aβ-O species, the method comprising:
providing a sample of tissue comprising cells;
microdissecting the cells and transferring the cells to an upper chamber of a manifold comprising a plurality of layered wells each comprising an upper chamber and a lower chamber, each chamber comprising independent fluidic connections and an adjustable valve separating the upper chambers and lower chambers;
assembling the manifold on an indium-titanium oxide coated glass slide;
introducing one or more anti-Aβ antibodies into the lower chamber of the layered well containing the cells in the upper chamber, incubating for a period of time, and washing the layered well;
opening the adjustable valve separating the upper chamber and lower chamber to permit the cells in the upper chamber to contact the one or more anti-Aβ antibodies in the lower chamber, incubating for a period of time, and washing the layered well to remove non-captured material;
introducing a matrix solution and allowing crystallization; and
removing the manifold and analyzing a co-crystallized sample using mass spectrometry to identify the presence of the Aβ-M and Aβ-O species.

2. The method of claim 1, wherein the tissue is human brain tissue comprising human brain cells.

3. The method of claim 1, wherein the manifold is comprised of a polymeric material comprising poly(dimethylsiloxane) (PDMS), polycarbonate (PC), poly-methyl-meta-acrylate (PMMA), cyclic olefin copolymer (COC), polyimide, or combinations thereof.

4. The method of claim 3, wherein the manifold is comprised of PDMS.

5. The method of claim 1, wherein the one or more anti-Aβ antibodies comprises an Aβ-specific antibody, an amyloid oligomer-specific antibody, or a combination thereof.

6. The method of claim 5, wherein the one or more anti-Aβ antibodies comprises an immunoglobulin G (IgG) 6E10 antibody.

7. The method of claim 1, wherein the matrix solution comprises α-cyano-4-hydroxycinnamic acid or sinapinic acid in acetonitrile and trifluoroacetic acid.

8. The method of claim 1, wherein the mass spectrometry comprises matrix-assisted laser desorption/ionization (MALDI) mass spectrometry.

9. The method of claim 1, wherein each layered well comprises a well area size ranging from about 50 μm×about 50 μm to about 500 μm×about 500 μm.

10. The method of claim 9, wherein each layered well comprises a well area size of about 500 μm×about 500 μm.

11. The method of claim 1, wherein microdissecting the cells comprises laser capture microdissection (LCM).

12. The method of claim 1, wherein each layered well comprises from about 1 to about 100 individual cells.

13. The method of claim 12, wherein each layered well comprises from about 1 to about 20 individual cells.

14. The method of claim 1, wherein the Aβ-M species comprise monomers of Aβ₁₋₄₂, Aβ₁₋₄₀, Aβ₁₋₃₉, Aβ₂₋₄₃, or combinations thereof.

15. The method of claim 1, wherein the Aβ-O species comprise oligomers of Aβ₁₋₄₂, Aβ₁₋₄₀, Aβ₁₋₃₉, Aβ₂₋₄₃, or combinations thereof.

16. The method of claim 15, wherein the oligomers of Aβ₁₋₄₂, Aβ₁₋₄₀, Aβ₁₋₃₉, Aβ₂₋₄₃, or combinations thereof comprise dimers, trimers, tetramers, pentamers, hexamers, heptamers, octamers, nonamers, decamers, 11-mers, 12-mers, 13-mers, 14-mers, 15-mers, 16-mers, 17-mers, 18-mers, 19-mers, 20-mers, or combinations thereof.

17. The method of claim 1, further comprising a bovine serum albumin (BSA) blocking step in the layered well prior to opening the adjustable valve.

18. The method of claim 1, wherein the method has a limit of detection for the Aβ-M and Aβ-O species of about 1.60×10⁸ to about 2.90×10¹¹ Aβ molecules per layered well.

19. A system for analyzing tissue for the presence of Aβ-M and Aβ-O species, the system comprising:
an apparatus for microdissection of cells from a sample of tissue;
a manifold comprising a plurality of layered wells each comprising an upper chamber and a lower chamber, each chamber comprising independent fluidic connections and an adjustable valve separating the upper chambers and lower chambers, wherein the manifold is assembled on an indium-titanium oxide coated glass slide;
one or more anti-Aβ antibodies positioned within the lower chamber of the layered well;
a matrix solution; and
a mass spectrometer.

20. The system of claim 19, wherein the sample of tissue is a sample of human brain tissue comprising human brain cells.

21. The system of claim 19, wherein the apparatus for microdissection comprises a laser capture microdissection (LCM) apparatus.

22. The system of claim 19, wherein the manifold is comprised of a polymeric material comprising poly(dimethylsiloxane) (PDMS).

23. The system of claim 19, wherein the one or more anti-Aβ antibodies comprises an Aβ-specific antibody, an amyloid oligomer-specific antibody, or a combination thereof.

24. The system of claim 23, wherein the one or more anti-Aβ antibodies comprises an immunoglobulin G (IgG) 6E10 antibody.

25. The system of claim 19, wherein the matrix solution comprises α-cyano-4-hydroxycinnamic acid or sinapinic acid in acetonitrile and trifluoroacetic acid.

26. The system of claim 19, wherein the mass spectrometer comprises a mass spectrometer configured for matrix-assisted laser desorption/ionization (MALDI) mass spectrometry.

27. A method for analyzing tissue for the presence of one or more protein biomarkers, the method comprising:
providing a sample of tissue comprising cells;
microdissecting the cells and transferring the cells to an upper chamber of a manifold comprising a plurality of layered wells each comprising an upper chamber and a lower chamber, each chamber comprising independent fluidic connections and an adjustable valve separating the upper chambers and lower chambers;
assembling the manifold on an indium-titanium oxide coated glass slide;
introducing one or more antibodies into the lower chamber of the layered well containing the cells in the upper chamber, incubating for a period of time, and washing the layered well;
opening the adjustable valve separating the upper chamber and lower chamber to permit the cells in the upper chamber to contact the one or more antibodies in the lower chamber, incubating for a period of time, and washing the layered well to remove non-captured material;
introducing a matrix solution and allowing crystallization; and
removing the manifold and analyzing a co-crystallized sample using mass spectrometry to identify the presence of the one or more protein biomarkers.
